(19) United States
(12) Patent Application Publication
(10) Pub. No.: US 2014/0106437 A1
(43) Pub. Date: Apr. 17, 2014

(54) REMOVING COMPOUNDS FROM WATER USING A SERIES OF REACTOR TUBES CONTAINING CATHODES COMPRISED OF A MIXED METAL OXIDE

(71) Applicants: Nicholas Eckelberry, Los Angeles, CA (US); Jose L. Sanchez, Los Angeles, CA (US)

(72) Inventors: Nicholas Eckelberry, Los Angeles, CA (US); Jose L. Sanchez, Los Angeles, CA (US)

(21) Appl. No.: 14/109,336

(22) Filed: Dec. 17, 2013

Related U.S. Application Data
(63) Continuation-in-part of application No. 13/942,348, filed on Jul. 15, 2013.
Application No. 13/942,348 is a continuation-in-part of application No. 13/872,044, filed on Apr. 26, 2013.
Application No. 13/872,044 is a continuation-in-part of application No. 13/865,087, filed on Apr. 17, 2013.
Application No. 13/865,087 is a continuation-in-part of application No. 13/753,484, filed on Jan. 29, 2013.
(60) Provisional application No. 61/592,522, filed on Jan. 30, 2012.
(60) Provisional application No. 61/625,463, filed on Apr. 17, 2012.
(60) Provisional application No. 61/649,083, filed on May 18, 2012.

(51) Int. Cl. C12M 1/26

(52) U.S. Cl. CPC C12M 33/14
USPC 435/257.1; 435/297.1

(57) ABSTRACT

A series of reactor tubes can be employed to remove a biofilm from a growth medium. Each reactor tube can contain a cathode comprised of a blend of transition metal oxides. As the growth medium is passed through the series of reactor tubes, a specific voltage and amperage can be applied to the reactor tubes to cause the biofilm to aggregate without damaging the algae. The aggregated biofilm can then be easily removed from the growth medium.

### CROSS-REFERENCE TO RELATED APPLICATIONS

This application is a continuation-in-art of U.S. patent application Ser. No. 13/942,348 filed on Jul. 15, 2013, titled Removing Ammonia From Water, which is a continuation-in-part of U.S. patent application Ser. No. 13/872,044 filed on Apr. 26, 2013, titled Producing Algae Biomass Having Reduced Concentration Of Contaminants, which is a continuation-in-part of U.S. patent application Ser. No. 13/865,087 filed Apr. 17, 2013, titled Harvesting And Dewatering Algae Using A Two-Stage Process, which is a continuation-in-part of U.S. patent application Ser. No. 13/753,484, filed Jan. 29, 2013, titled Systems And Methods For Harvesting And Dewatering Algae, which claims priority to U.S. Provisional Patent Application No. 61/592,522, filed Jan. 30, 2012, titled Systems And Methods For Harvesting And Dewatering Algae.

U.S. patent application Ser. No. 13/865,087 also claims priority to U.S. Provisional Patent Application No. 61/625,463, filed Apr. 17, 2012, titled Solute Extraction From An Aqueous Medium Using A Modular Device, and to U.S. Provisional Patent Application No. 61/649,083, filed May 28, 2012, titled Modular Systems And Methods For Extracting A Contaminant From A Solution. This application claims the benefit of these two provisional applications.

The disclosures of each of the applications to which the present application claims priority are incorporated by reference.

### BACKGROUND

Algae are currently being grown for use in various different industries including the biofuel, pharmaceutical, and algae-to-food industries. For algae to grow successfully, a suitable growth medium must be maintained. One major problem that algae growers face is the buildup of a biofilm within the growth medium. A biofilm comprises the buildup of invasive species such as bacteria, low value filamentous algae, and protein strands. The biofilm interferes with growth in various ways including: by utilizing resources such as CO₂ and food that the algae require fro growth; by blocking light from reaching the algae thereby limiting photosynthesis; in the case of bacteria, by directly attacking the algae cells; and by creating anaerobic conditions in the growth medium which can result in the production of harmful substances.

Many techniques have been employed to attempt to address the biofilm problem. These techniques include the use of bio-engineered algae, modified feeding regimens, chemicals, and changes to the salinity or acidity of the growth medium. In many cases, these techniques do not produce desirable results or may require an unsatisfactory amount of time to correct a biofilm buildup.

One particular device that is commonly used to reduce biofilm buildup in municipal water treatment facilities and public aquariums is a protein skimmer. A protein skimmer functions by using the polarity of proteins to cause the proteins to accumulate. Due to their intrinsic charge, water-borne proteins are either repelled or attracted by the air/water interface. Accordingly, protein skimmers generate a large air/water interface by injecting large numbers of bubbles into the water. By increasing the number of bubbles in the water (and particularly, the number of small bubbles), the total surface area of the air/water interface is increased thereby creating a larger interface to which the proteins may attach. The end result is that the organic and inorganic compounds aggregate at the air/water interface and eventually float to the surface where they can be skimmed from the water.

Although protein skimmers are effective at removing a biofilm, they require a substantial amount of time to do so. In particular, the process of forming sufficient bubbles, attracting the compounds to the bubbles, and floating the compounds to the surface can require an unsatisfactory amount of time for many applications.

### BRIEF SUMMARY

The present invention is generally directed to employing a series of reactor tubes to remove a biofilm from an algae growth medium. Each reactor tube can contain a cathode comprised of a blend of transition metal oxides. As the growth medium is passed through the series of reactor tubes, a specific voltage and amperage can be applied to the reactor tubes to cause the biofilm to aggregate without damaging the algae. The aggregated biofilm can then be easily removed from the growth medium.

The system can therefore, in essence, function as a protein skimmer. However, the system does not require the generation of micro-bubbles to effectuate the aggregation of the biofilm. In contrast, the system of the present invention can immediately cause the aggregation of the biofilm via the ionic exchange that occurs at the mixed metal oxide cathode. By employing specific voltages and amperages, the biofilm can be caused to aggregate without damaging the algae. A further benefit of this process is that the applied voltages and amperages can kill bacteria present within the growth medium. As a result, after passing through the system of the present invention, the growth medium can be free of the biofilm and bacteria leaving an environment ideally suited for continued growth of the algae.

In one embodiment, the present invention is implemented as a system for removing a biofilm from a growth medium. The system comprises a plurality of reactor tubes connected in series, each reactor tube comprising an outer anode and an inner cathode being positioned centrally within the outer anode such that a spacing between 3 mm and 10 mm exists between the outer surface of the cathode and the inner surface of the anode, each inner cathode comprising a mixed metal oxide. The system also comprises a pump connected to an input of the plurality of reactor tubes, the pump configured to receive a growth medium having a biofilm and to pump the growth medium through the series of reactor tubes; a power supply for supplying a voltage differential to the anode and the cathode in each reactor tube, the voltage differential causing the generation of ions within the growth medium, the ions causing compounds within the biofilm to aggregate into an aggregated biofilm; and a filter for removing the aggregated biofilm from the growth medium.

In another embodiment, the present invention is implemented as a method for removing a biofilm from a growth medium. A growth medium having a biofilm is supplied to one or more reactor tubes, each reactor tube comprising an outer anode and an inner cathode being positioned centrally within the outer anode such that a spacing between 3 mm and 10 mm exists between the outer surface of the cathode and the inner surface of the anode, each inner cathode comprising a mixed metal oxide. A voltage differential is supplied between the anode and the cathode in each reactor tube to cause the generation of ions within the growth medium, the ions causing compounds within the biofilm to aggregate into an aggregated biofilm. The aggregated biofilm is then filtered from the growth medium.

This summary is provided to introduce a selection of concepts in a simplified form that are further described below in the Detailed Description. This Summary is not intended to identify key features or essential features of the claimed subject matter.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates an example system for removing a biofilm from a growth medium using a series of reactor tubes employing MMO cathodes;

FIG. 2A illustrates a cross-sectional front view of a reactor tube containing an inner cathode centered between an outer anode; and

FIG. 2B illustrates a cross-sectional side view of the reactor tube.

### DETAILED DESCRIPTION

The present invention is generally directed to employing a series of reactor tubes to remove a biofilm from an algae growth medium. Each reactor tube can contain a cathode comprised of a blend of transition metal oxides. As the growth medium is passed through the series of reactor tubes, a specific voltage and amperage can be applied to the reactor tubes to cause the biofilm to aggregate without damaging the algae. The aggregated biofilm can then be easily removed from the growth medium.

The system can therefore, in essence, function as a protein skimmer. However, the system does not require the generation of micro-bubbles to effectuate the aggregation of the biofilm. In contrast, the system of the present invention can immediately cause the aggregation of the biofilm via the ionic exchange that occurs at the mixed metal oxide cathode. By employing specific voltages and amperages, the biofilm can be caused to aggregate without damaging the algae. A further benefit of this process is that the applied voltages and amperages can kill bacteria present within the growth medium. As a result, after passing through the system of the present invention, the growth medium can be free of the biofilm and bacteria leaving an environment ideally suited for continued growth of the algae.

In one embodiment, the present invention is implemented as a system for removing a biofilm from a growth medium. The system comprises a plurality of reactor tubes connected in series, each reactor tube comprising an outer anode and an inner cathode being positioned centrally within the outer anode such that a spacing between 3 mm and 10 mm exists between the outer surface of the cathode and the inner surface of the anode, each inner cathode comprising a mixed metal oxide. The system also comprises a pump connected to an input of the plurality of reactor tubes, the pump configured to receive a growth medium having a biofilm and to pump the growth medium through the series of reactor tubes; a power supply for supplying a voltage differential to the anode and the cathode in each reactor tube, the voltage differential causing the generation of ions within the growth medium, the ions causing compounds within the biofilm to aggregate into an aggregated biofilm; and a filter for removing the aggregated biofilm from the growth medium.

In another embodiment, the present invention is implemented as a method for removing a biofilm from a growth medium. A growth medium having a biofilm is supplied to one or more reactor tubes, each reactor tube comprising an outer anode and an inner cathode being positioned centrally within the outer anode such that a spacing between 3 mm and 10 mm exists between the outer surface of the cathode and the inner surface of the anode, each inner cathode comprising a mixed metal oxide. A voltage differential is supplied between the anode and the cathode in each reactor tube to cause the generation of ions within the growth medium, the ions causing compounds within the biofilm to aggregate into an aggregated biofilm. The aggregated biofilm is then filtered from the growth medium.

In U.S. patent application Ser. No. 13/942,348 (the '348 application), a system for removing ammonia from wastewater is described. The system employs a series of reactor tubes that include a centrally positioned anode or cathode comprised of a mixed metal oxide (MMO). The use of the MMO anode or cathode resulted in the increased production of free chlorine to assist in the breakdown of ammonia. It has been found that, by using specific voltages and currents, a system similar to the system described in the '348 application can be used to cause the aggregation of a biofilm without damaging the algae. In particular, the series of reactor tubes employed in the system of the '348 application can be powered at specific voltages and amperages to cause the aggregation of a biofilm. Accordingly, the present invention can be implemented in systems similar to those described in the '348 application. However, the present invention can also be implemented in other more general systems. An example of such a system is provided below.

System for Aggregating and Removing a Biofilm from a Growth Medium

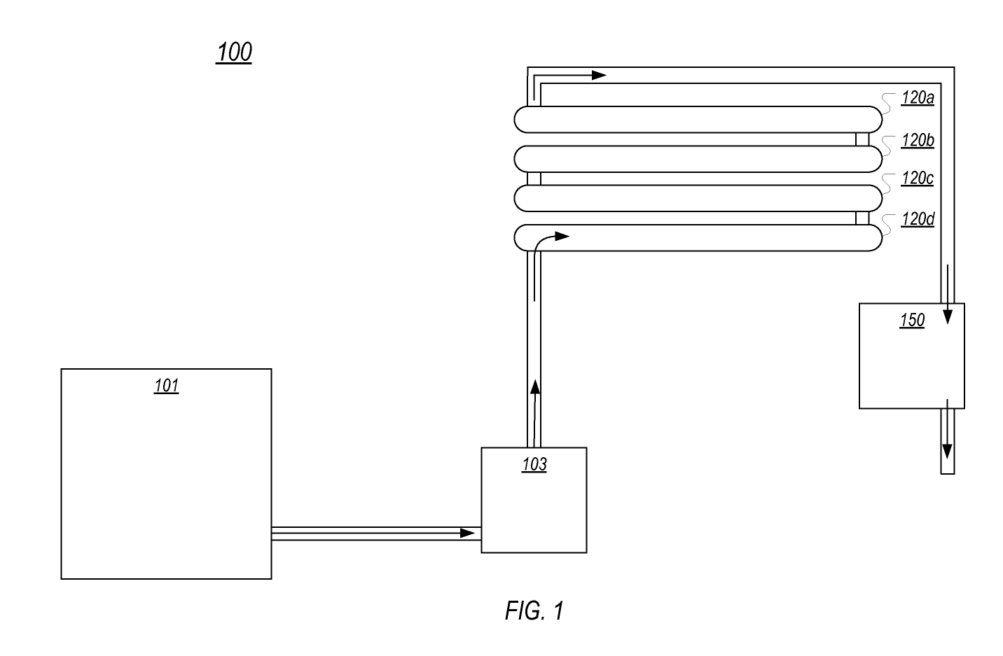

FIG. 1 illustrates an example system 100 that can be used to aggregate and remove a biofilm from a growth medium. System 100 includes a growth medium source 101 that supplies the growth medium containing the biofilm. Growth medium source 101 can be any source of a growth medium including a pond or tank. The growth medium can be fed through a series of reactor tubes 120a-120d (which will generally be identified with 120). Although four reactor tubes are shown in system 100, other numbers of reactor tubes can be used to accomplish a desired level of biofilm aggregation. In some embodiments, two reactor tubes in series may be sufficient.

Each reactor tube 120 is comprised of an outer anode forming the tube shape and an inner cathode positioned centrally within the tube. The inner cathode is positioned no more than 10 mm from the outer anode. In some embodiments, the outer anode can be comprised of stainless steel and the inner cathode can be comprised of a MMO. Although this description refers to the inner electrode as the cathode and the outer electrode as the anode, the anode and cathode could also be reversed.

MMOs are compounds composed of oxygen atoms bound to transition metals. MMOs have a wide variety of surface structures which affect the surface energy of these compounds and influence their chemical properties. The relative acidity and basicity of the atoms present on the surface of metal oxides is also affected by the coordination of the metal cation and oxygen anion, which alter the catalytic properties of these compounds.

In some embodiments, the blend of MMOs that can be used to cause the ionic exchange to destroy nuclei free species and aggregate or flocculate the biofilm can be a blend of the six platinum group metals layered onto a titanium core. These metals include ruthenium, rhodium, palladium, osmium, iridium, and platinum. A particular blend of metals can be created for a desired result. For example a weighted blend of ruthenium will generate a plurality of protons whereas weighing the blend towards iridium will generate a plurality of hydroxyls. In any case, the biofilm is aggregated or flocculated by cationic or anionic reactions.

Unlike protein skimmers which employ micro-bubbles to create a large air/water interface to attract protein-based compounds, the system of the present invention can cause the protein-based compounds (or biofilm) to attract and aggregate without the production of micro-bubbles. In other words, the system of the present invention does not require a source of bubbles. In contrast, as described above, the flow of an electric current through the MMO cathode produces free ions which interact with the compounds in the biofilm to cause the biofilm to aggregate together. These interactions occur immediately as the growth medium passes through the reactor tubes resulting in quick aggregation of the biofilm. The biofilm can therefore be aggregated sufficiently for removal as the growth medium exits the series of reactor tubes.

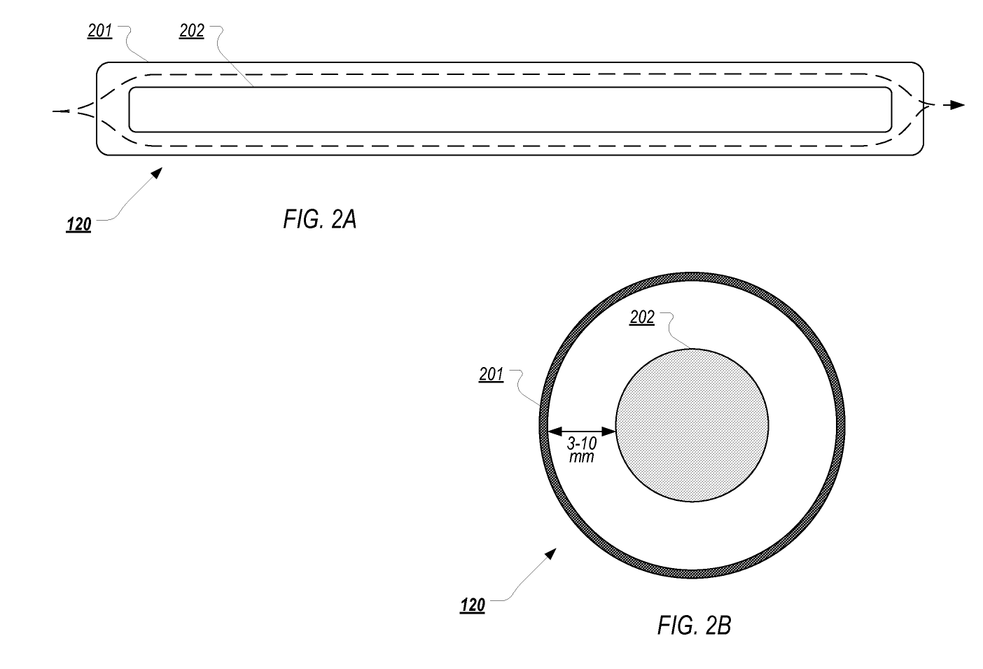

FIGS. 2A and 2B illustrate a cross-sectional front and side view respectively of a reactor tube 120. As shown in FIG. 2A, an inner cathode 202 is centrally positioned within an outer anode 201 to create a narrow pathway around the circumference of cathode 202 through which the growth medium can flow (as indicated by the arrow) Inner cathode 202 is coated with a MMO.

As shown in FIG. 2B, in some embodiments, the spacing between anode 201 and cathode 202 can be between 3 and 10 mm with an optimal exact spacing being dependent on the conductivity of the growth medium. A spacing within the 3-10 mm range has proven to be optimal for causing the necessary cationic or anionic reactions to aggregate the biofilm.

Returning to FIG. 1, after the growth medium has passed through the series of reactor tubes 120, a strainer 150 can be used to remove the aggregated biofilm from the growth medium. The growth medium can then be routed back to the source 101 or diverted to another location (e.g. for algae harvesting, growth, etc.).

The ideal voltage and amperage will vary based on the conductivity of the growth medium. When the growth medium has a conductivity between 0 and 2000 mS, 15 volts and between 5 to 10 amps (DC) is suitable. When the growth medium has a conductivity between 200 and 10,000 mS, 10 volts and 5 amps (DC) is suitable. When the growth medium has a conductivity between 10,000 and 20,000 mS, 5 volts and less than 3 amps (DC) is suitable. Finally, when the growth medium has a conductivity in excess of 20,000 mS, 1.3 volts and 1 amp (DC) is suitable.

The electrodes (i.e. anode 201 and cathode 202) can be made of a metal, composite, or other material known to impart conductivity, such as, but not limited to silver, copper, gold, aluminum, zinc, nickel, brass, bronze, iron, lead, platinum group metals, steel, stainless steel, carbon allotropes, and/or combinations thereof. Non-limiting examples of conductive carbon allotropes can include graphite, graphene, synthetic graphite, carbon fiber (iron reinforced), nano-carbon structures, and other form of deposited carbon on silicon substrates.

Example Test Results

In a first test, a 200 gallon growth medium of a photo-bioreactor growing Scenedesmus that showed extensive rotifer contamination was processed through a system in accordance with the present invention. The system was powered at 10 volts and 10 amps (DC) while the 200 gallons were pumped through the reactor tubes. The growth medium was then passed through a 5 micron sieve. Prior to treatment, the growth medium has an algae growth yield of 80 mg/liter. Within 48 hours after treatment, the yield had increased to 120 mg/liter in 48 hours. A microscope analysis of the post treatment growth medium showed an absence of rotifers

In a second test, a 2000 liter open pond of fresh water Haematococcus pluvialis was divided in two ponds and fed with the same amount of nutrients. The two ponds were adjacent one another and subject to the same environmental conditions. One of the ponds was left untreated while the other pond was treated by passing its whole volume through the system. The system was powered at 7.5 volts and 10 amps (DC) at a flow of 4 liters per minute every 6 hours. The untreated tank did not present growth for two days and crashed by bacterial attack. The treated tank presented an average net algae growth rate of 50 mg/L the first day, 87 mg/L the second day, and 113 mg/L the third day and did not crash.

In a third test, 200 gallons of salt water Nanochloropsis was processed using the system of the present invention. The system was powered at 1.2 volts and 0.02 amps (DC). Within 48 hours of treatment, the growth yield had increased by 50%.

From each of these tests, it was concluded that the increase in growth of the algae was due to the reduction in the biofilm and/or harmful species within the growth medium. By removing the biofilm, a greater amount of CO₂, nutrients, and sunlight were accessible to the algae leading to an increased growth rate. As can be seen, this increase in growth occurred almost immediately after treatment and continued as long as the biofilm was prevented from building up (e.g. by retreating the growth medium). The system of the present invention therefore provides a quick and efficient means to enhance the growth of an algae biomass by removing a biofilm.

Additional lab results are included as Appendix A below. These results indicate a reduction in bacterial load across all experimental variations including complete bacteria elimination in some variations.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed:

1. A system for removing a biofilm from a growth medium comprising:
a plurality of reactor tubes connected in series, each reactor tube comprising an outer anode and an inner cathode being positioned centrally within the outer anode such that a spacing between 3 mm and 10 mm exists between the outer surface of the cathode and the inner surface of the anode, each inner cathode comprising a mixed metal oxide;
a pump connected to an input of the plurality of reactor tubes, the pump configured to receive a growth medium having a biofilm and to pump the growth medium through the series of reactor tubes;
a power supply for supplying a voltage differential to the anode and the cathode in each reactor tube, the voltage differential causing the generation of ions within the growth medium, the ions causing compounds within the biofilm to aggregate into an aggregated biofilm; and
a filter for removing the aggregated biofilm from the growth medium.

2. The system of claim 1, wherein the outer anode comprises stainless steel.

3. The system of claim 1, wherein the mixed metal oxide comprises a blend of platinum group metals layered onto a titanium core.

4. The system of claim 1, wherein the blend of platinum group metals comprises two or more of ruthenium, rhodium, palladium, osmium, iridium, and platinum.

5. The system of claim 1, wherein the mixed metal oxide comprises ruthenium such that protons are generated when the voltage differential is applied.

6. The system of claim 1, wherein the mixed metal oxide comprises iridium such that hydroxyls are generated when the voltage differential is applied.

7. The system of claim 1, wherein the ions comprise cations.

8. The system of claim 1, wherein the ions comprise anions.

9. The system of claim 1, wherein the voltage differential is between 1.3 and 15 volts.

10. The system of claim 1, wherein the voltage differential is selected to generate a current between 1 and 5 amps.

11. The system of claim 1, wherein the voltage differential is selected to generate a current of approximately 1.5 amps.

12. The system of claim 1, wherein the voltage differential is selected to generate a current of approximately 5 amps.

13. The system of claim 1, wherein the power supply is a DC power supply.

14. The system of claim 1, wherein the voltage differential is supplied for at least 30 minutes.

15. A method for removing a biofilm from a growth medium comprising:
supplying a growth medium having a biofilm to one or more reactor tubes, each reactor tube comprising an outer anode and an inner cathode being positioned centrally within the outer anode such that a spacing between 3 mm and 10 mm exists between the outer surface of the cathode and the inner surface of the anode, each inner cathode comprising a mixed metal oxide;
supplying a voltage differential between the anode and the cathode in each reactor tube to cause the generation of ions within the growth medium, the ions causing compounds within the biofilm to aggregate into an aggregated biofilm; and
filtering the aggregated biofilm from the growth medium.

16. The method of claim 15, wherein the mixed metal oxide comprises a blend of platinum group metals layered onto a titanium core.

17. The method of claim 15, wherein the voltage differential is selected to generate a current between 1 and 5 amps.

18. A system for removing a biofilm from a growth medium comprising:
a plurality of reactor tubes connected in series, each reactor tube comprising an outer anode and an inner cathode being positioned centrally within the outer anode such that a spacing between 3 mm and 10 mm exists between the outer surface of the cathode and the inner surface of the anode, each inner cathode comprising a coating consisting of a blend of platinum group metals;
a pump connected to an input of the plurality of reactor tubes, the pump configured to receive a growth medium having a biofilm and to pump the growth medium through the series of reactor tubes;
a power supply for causing a current between 1 and 5 amps to flow between the anode and cathode of each reactor tube thereby causing the generation of ions within the growth medium, the ions causing compounds within the biofilm to aggregate into an aggregated biofilm; and
a filter for removing the aggregated biofilm from the growth medium.

19. The system of claim 18, wherein the current is generated using a voltage differential between 1.3 and 15 volts.

20. The system of claim 18, wherein the growth medium contains algae.
